(19) United States
(12) Patent Application Publication
(10) Pub. No.: US 2025/0242309 A1
(43) Pub. Date: Jul. 31, 2025

(54) APPARATUS AND METHOD FOR SEPARATING ORGANIC ACID ALKALI SALTS INTO ORGANIC ACIDS AND ALKALI SALTS BY USING DIFFUSION DIALYSIS

(71) Applicant: KOREA RESEARCH INSTITUTE OF CHEMICAL TECHNOLOGY, Daejeon (KR)

(72) Inventors: Young Kyu HWANG, Daejeon (KR); Ji Hoon KIM, Daejeon (KR); Changho YOO, Daejeon (KR); In Hyoup SONG, Daejeon (KR); Dong Won HWANG, Daejeon (KR); Gwang Nam YUN, Daejeon (KR); Seung Hyeok CHA, Daejeon (KR); Kyung Ryul OH, Daejeon (KR)

(73) Assignee: KOREA RESEARCH INSTITUTE OF CHEMICAL TECHNOLOGY, Daejeon (KR)

(21) Appl. No.: 19/088,403

(22) Filed: Mar. 24, 2025

Related U.S. Application Data
(63) Continuation of application No. PCT/KR2023/017977, filed on Nov. 9, 2023.

(30) Foreign Application Priority Data
Nov. 21, 2022 (KR) 10-2022-0156058
Dec. 22, 2022 (KR) 10-2022-0181654

(51) Int. Cl. B01D 61/24; B01D 69/02; C07C 51/47

(52) U.S. Cl. CPC B01D 61/243; B01D 69/02; C07C 51/47; B01D 2325/14; B01D 2325/42

(57) ABSTRACT

Disclosed is a means for facilitating, in a method for separating and individually collecting organic acids and alkali salts from organic acid alkali salts, separation/collection of lactic acids and alkali salts via an economic and eco-friendly diffusion analysis method without adding a complicated process such as electrodialysis or electrolysis. During a process of diffusion dialysis of organic acid salts, gaseous components that can release protons are injected into a collection part so that the electroneutrality in solution can be maintained and protons can be transferred to a feed part, thereby facilitating the collection of organic acids. Also, alkali cations that have moved to the collection part may form alkali salts with the gaseous components and be then collected, and a nonsolvent may be used to lower the solubility of the alkali salts for easy collection.

### CROSS-REFERENCE TO RELATED APPLICATIONS

This application is a continuation of PCT International Patent Application No. PCT/KR2023/017977 which has an International filing date of Nov. 9, 2023, and which claims priority to Korean Patent Application No. 10-2022-0156058 filed Nov. 21, 2022, and Korean Patent Application No. 10-2022-0181654 filed Dec. 22, 2022, the entire contents of each of which are incorporated herein by reference.

### TECHNICAL FIELD

The present disclosure relates to an apparatus and method for separating organic acid alkali salts into organic acids and alkali salts by using the principle of diffusion dialysis. More specifically, the present disclosure relates to a separation apparatus and method that can promote an effect of diffusion dialysis by using a diffusion dialysis method through a cation exchange membrane without using electricity, heat, or expensive chemicals, and by bubbling at least one gas selected from the group consisting of carbon dioxide, sulfur dioxide, and nitrogen oxides in a recovery unit.

### BACKGROUND ART

Organic acids are acidic substances widely present in natural organisms, and they include organic compounds with functional groups that can generate hydrogen ions (H⁺), such as carboxyl groups, sulfonic acid groups, alcohol groups, thiol groups, and enol groups. In general, organic acids containing a carboxyl group (—COOH) such as lactic acids, acetic acids, and formic acids are sometimes called organic acids.

Industrially, the organic acids containing the carboxyl group are produced through fermentation, which is a process of obtaining energy by oxidizing sugar nutrients in the internal environment of microorganisms. Alternatively, the organic acids containing the carboxyl group are produced through a chemical oxidation process under specific conditions. The organic acids containing the carboxyl group have a wide variety of uses depending on the number of carbon atoms and structure of the produced organic acids.

Lactic acids, which are produced by inorganic respiration of sugar, are substances with a variety of uses ranging from additives in the food field, preservatives, animal feed, and building blocks in the chemical field. Especially as lactic acids are used as a main raw material for poly lactic acid (PLA), which is a biodegradable plastics, the demand for lactic acids has increased.

To improve yield of organic acids such as lactic acids, final products are generally produced in a base environment regardless of any processes and exist in the form of alkali salts. Therefore, to recover the finally produced alkali salts into organic acids, the finally produced alkali salts are required to be treated with acid, and if fractional distillation is necessary, alcohol is added thereto for esterification. In this case, the problem is that this conventional production method consumes a large amount of heat energy and materials such as acid or alcohol to separate the lactic acids after production.

Accordingly, to address this problem, separation/recovery methods using membranes are being studied. In particular, cases of using a bipolar membrane electrodialysis method or diffusion dialysis method are emerging.

The bipolar membrane electrodialysis method is a separation/recovery method in which a cation exchange membrane and/or anion exchange membrane are placed between two bipolar membranes, and then an electric field is applied from the outside of the bipolar membrane. By this method, ions in a feed solution move under electrical attraction and pass through the ion exchange membranes.

China Registered Patent Publication No. 108385129 (published on Aug. 10, 2018) discloses a method of separating sodium formates produced by electrochemical reaction into formic acids and sodium hydroxides using the bipolar membrane electrodialysis method. Japanese Registered Patent Publication No. 6717935 (published on 2021 Aug. 2) discloses a bipolar membrane electrodialysis method and bipolar membrane electrodialysis system for purifying organic acids from aqueous solutions containing organic acid salts. These techniques can separate acids and bases without any special chemical reactions and have high selectivity and yield. However, these techniques have issues such as requiring use of high-density electrical energy and having a complicated structure.

Meanwhile, the diffusion dialysis method is a generally used separation method to separate acids or bases in a wastewater purification system. That means, the diffusion dialysis method refers to a separation/recovery method, in which a cation or anion exchange membrane is placed in the middle, and a feed solution is injected into one side while a recovery solution is injected into the other side. In this case, a concentration gradient is formed around a cation or anion exchange membrane. On the basis of the concentration gradient, ions move from the feed solution to the recovery solution, thereby only cations or anions are selectively separated. The diffusion dialysis method is performed on the basis of a simple structure, making it easy to scale up the process. In addition, the diffusion dialysis method does not require additional energy, making separation and recovery more environmentally friendly. However, in the diffusion dialysis method, separation is relatively slow, and also as diffusion progresses, the concentration gradient decreases, and as a result, the separation becomes even slower. Due to that, the diffusion dialysis method is generally performed in combination with other processes. For example, Korea Patent No. 10-2003918 (published on Jun. 5, 2019) discloses a technique regarding a separation method of sugar and acid through electrolysis. This technique regarding the separation of sugar and acid has the characteristic of requiring reduced energy for electrolysis by primarily separating the acid through a diffusion dialysis process.

However, there is a need to develop a technique that can efficiently separate/recover organic acids and alkali salts from organic acid alkali salts while still using only the eco-friendly diffusion dialysis method.

### RELATED ART DOCUMENTS

(Patent Document 0001) Chinese Patent No. 108385129 (published on Aug. 10, 2018)

(Patent Document 0002) Japanese Patent No. 6717935 (published on Aug. 2, 2021)

(Patent Document 0003) Korean Patent No. 10-2003918 (published on Jun. 5, 2019)

### DISCLOSURE

### Technical Problem

The objective of the present disclosure is to provide an apparatus and method for separating organic acid alkali salts into organic acids and alkaline salts in both an environmentally friendly and efficient way by improving the conventional diffusion dialysis method.

### Technical Solution

One embodiment of the present disclosure for achieving the objective may disclose an apparatus for separating organic acid alkali salts into organic acids and alkali salts. The apparatus for separating organic acids alkali salts into organic acids and alkali salts may include: a diffusion dialysis section divided into a feed unit and a recovery unit by a cation exchange membrane; a feed injection section for injecting a feed solution containing organic acid alkali salts into the feed unit; a recovery solution injection section for injecting a recovery solution containing water into the recovery unit; and a gas injection means for injecting at least one gas selected from the group consisting of carbon dioxide, sulfur dioxide, and nitrogen oxide into the solution in the recovery unit.

According to the embodiment, the cation exchange membrane may have at least one functional group selected from the group consisting of —COOH, —OH, and —SO₃H.

Also, according to the embodiment, a means for disturbing a fluid flow may be further provided in the feed unit and the recovery unit, respectively.

Also, according to the embodiment, fluid flow directions in the feed unit and the recovery unit may be opposite to each other or may be the same.

In addition, the apparatus for separating organic acid alkali salts into organic acids and alkali salts according to the embodiment may be further provided with a separation means for separating the alkaline salts generated in the recovery unit into a recovery solution.

Another embodiment of the present disclosure may disclose a method of separating organic acid alkali salts into organic acids and alkali salts. The method may include: injecting a feed solution containing organic acid alkali salts into the feed unit of an apparatus, which has a diffusion dialysis section divided into a feed unit and a recovery unit by a cation exchange membrane and injecting a recovery solution containing water into the recovery unit, and injecting at least one gas selected from the group consisting of carbon dioxide, sulfur dioxide, and nitrogen oxides into the recovery unit, thereby promoting diffusion dialysis of alkaline cations through the cation exchange membrane.

The other embodiment may further include recovering the alkaline salts obtained in the recovery unit from the recovery solution.

According to the other embodiment, the gas may be carbon dioxide, and the alkali salts generated may be alkali carbonates and/or alkali bicarbonates.

According to the other embodiment, the diffusion dialysis section may have a temperature in a range of 2° C. to 80° C.

Also, according to the other embodiment, the recovery unit may have a pH in the range of 2 to 7.

### Advantageous Effects

The present disclosure uses an economical and environmentally friendly diffusion dialysis method to separate organic acid alkali salts into organic acids and alkali salts, and at the same time, the present disclosure involves adding a predetermined gas to the recovery unit using a method such as bubbling or pressurization without adding complex processes such as electrodialysis or electrolysis. This can promote diffusion dialysis. The predetermined gas is a gas corresponding to greenhouse gas or exhaust gas, and the predetermined gas can be utilized in a diffusion dialysis system through bubbling or pressurization, thereby enabling the removal of the gas. Therefore, the present disclosure corresponds to an energy-efficient and environmentally friendly process.

In particular, when carbon dioxide gas is used for the bubbling, potassium bicarbonate or potassium carbonate is formed and precipitated, so the precipitate can be easily recovered. Furthermore, since potassium ion concentration in the recovery solution can be maintained at a low level, the present disclosure is excellent in maintaining a concentration gradient for diffusion dialysis. In addition, the alkali salts generated in the recovered solution can be recycled in a lactate production process or as a useful material, so the present disclosure can reduce costs.

### DESCRIPTION OF DRAWINGS

FIG. 1 schematically shows an apparatus for separating organic acid salts according to an embodiment of the present disclosure;

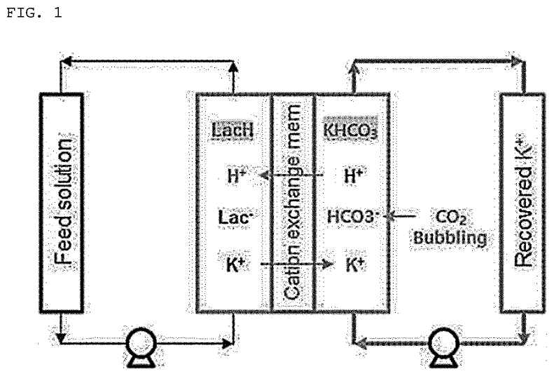

FIGS. 2(a) to 2(c) show graphs of measured flux values of potassium ions passing through the cation exchange membrane and of any organic acid anions selected from the group consisting of (a) formate ions, (b) lactate ions, and (c) gluconic acid ions according to Examples 1 to 3 and Comparative Examples 1 to 3;

FIGS. 3(a) to 3(c) show graphs of measured pH values of feed solution and recovery solution (permeate) in each of Examples 1 to 3 and Comparative Examples 1 to 3 depending on dialysis time;

FIGS. 4(a) to 4(c) show graphs of changes in measured potassium ion concentration depending on the dialysis time of feed solution and recovery solution (permeate) while performing diffusion dialysis according to Examples 1 to 3 and Comparative Examples 1 to 3;

FIGS. 5(a) to 5(c) show photographs of experiment results of alkaline (bi)carbonate precipitated from the permeate solution of Example 1 by using (a) acetone, (b) isopropanol, and (c) ethanol as exemplary non-solvents, respectively;

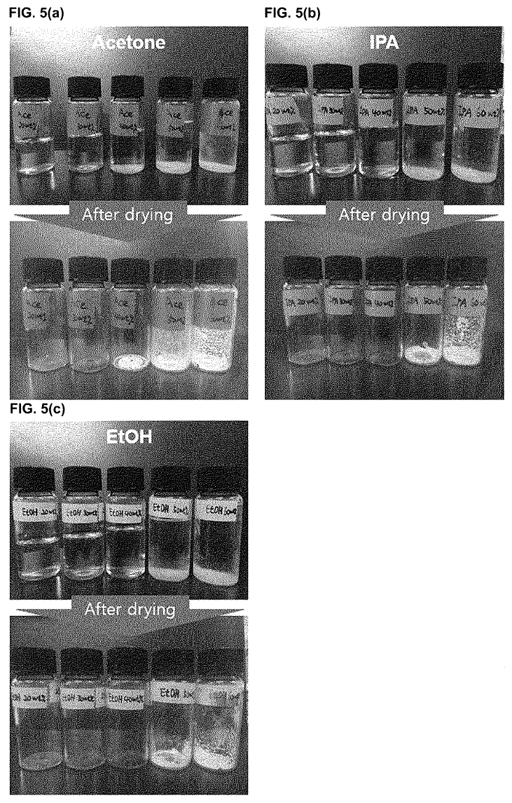

FIGS. 6(a) and 6(b) show (a) a graph of conductivity of feed solution and weight of the precipitated solids depending on a non-solvent content ratio, and (b) a graph of a recovery rate of K⁺ solution, which is back-estimated from a decrease in conductivity, respectively, when precipitating the permeate solution of Example 1 using each of the exemplary non-solvents; and

FIGS. 7(a) and 7(b) show a photograph and XRD spectrum of the solids precipitated by adding each of the non-solvents.

### BEST MODE

Throughout the specification herein, “organic acids” refer to organic compounds with functional groups capable of generating hydrogen ions (H⁺), including carboxyl groups, sulfonic acid groups, alcohol groups, thiol groups, and enol groups.

In addition, throughout the specification herein, “organic acid salts” refer to “compounds of ionic bonded organic acid anions (Lactate) and alkali metals or compounds of ionic bonded organic acid anions (Lactate) and ammonium cations.”

Throughout the specification herein, when a part is said to “include” a certain component, this means that it may further include other components rather than excluding other components unless specifically stated to the contrary.

Unless otherwise defined, all technical and scientific terms used in this specification have the same meaning as commonly understood by those skilled in the art to which the present disclosure pertains. In general, the nomenclature used herein is well-known and commonly used in the art.

The present disclosure provides an apparatus for separating organic acid alkali salts into organic acids and alkali salts. The apparatus for separating organic acids alkali salts into organic acids and alkali salts includes: a diffusion dialysis section divided into a feed unit and a recovery unit by a cation exchange membrane; a feed injection section for injecting a feed solution containing organic acid alkali salts into the feed unit; a recovery solution injection section for injecting a recovery solution containing water into the recovery unit; and a gas injection means for injecting at least one gas selected from the group consisting of carbon dioxide, sulfur dioxide, and nitrogen oxide into the recovery solution in the recovery unit.

In the organic acid alkali salts, the organic acids means organic acid salts that dissociate into anions in solution. The organic acids may include lactate salts, which are a product of lactic acids dissociated into anionic form in aqueous solutions. The organic acids are not limited as long as the organic acids are organic acid salts derived from organic compounds.

In addition, the organic acids contain at least one of linear and branched chain hydrocarbons having 1 to 30 carbon atoms, monocyclic or polycyclic hydrocarbons having 6 to 30 carbon atoms, and aromatic hydrocarbons having 6 to 30 carbon atoms, in addition to hydrogen ions.

The diffusion dialysis section is a container with an internal space. The internal space is divided into spaces for a feed unit and a recovery unit by a cation exchange membrane. At this time, the cation exchange membrane may be configured to be one or more. The cation exchange membrane may be used in one or more of the types below: flat-sheet type or hollow fiber type. The flat-sheet type may be scaled up in the form of spiral wound, stack, and plate-frame modules. The hollow fiber type may be used in the form of one or more of the honeycomb-type or submerged-type modules. One side of the cation exchange membrane comes into contact with the feed unit, and the other side comes into contact with the recovery unit.

When the cation exchange membrane takes flat-sheet type and there are two cation exchange membranes, the internal space may be divided into three spaces by the cation exchange membranes. The divided internal spaces alternately form the feed unit and the recovery unit. For example, when the leftmost space of the internal space is for the feed unit, the internal space of the container forming the diffusion dialysis section may be made from the feed unit/recovery unit/feed unit. When the leftmost space is for the recovery unit, the internal space of the container forming the diffusion dialysis section may be made from the recovery unit/feed unit/recovery unit.

The shape of the container forming the diffusion dialysis section is not limited to a cylindrical or polygonal shape and may be any shape, as long as the container has an internal space, and a cation exchange membrane may be installed therein. The material of the container is not particularly limited as long as the container may withstand any impact from the feed solution or recovery solution.

In addition, the cation exchange membrane may be a cation exchange membrane having at least one functional group selected from the group consisting of —COOH, —OH, and —SO₃H. The cation exchange membrane may preferably be a cation exchange membrane containing —SO₃H and may be made of a polymer material. The polymer material is not limited thereto but may be formed into a membrane using at least one selected from the group consisting of polyarylene ether sulfone (PAES), polyetheretherketone (PEEK), polyphenylsufide (PPS), polyphenyloxide (PPO), polyimide (PI), and polytetrafluoroethylene (PTFE).

The feed unit, which is one of the units divided by the cation exchange membrane, corresponds to a part where the feed solution containing organic acid alkali salts is injected.

The feed unit may have a temperature of 2° C. to 80° C. When the temperature inside the feed unit is lower than 2° C. or higher than 80° C., the state of the solution may change, or the cation exchange membrane may be destroyed.

The feed solution is injected into the feed unit through the feed injection section. The feed solution contains organic acid alkali salts and may be a product of fermentation or chemical reaction. Alkaline cations in the injected feed solution permeate and diffuse through the cation exchange membrane and move to the recovery unit. Meanwhile, organic acid anions may not penetrate the cation exchange membrane and remain in the feed solution. On the basis of this principle, it is possible to separate only the alkaline cations from the organic acid alkali salts.

The concentration of alkaline cations in the feed solution flowing out of the feed unit is lower than that in the feed solution before flowing into the feed unit. Depending on the area of the cation exchange membrane, a circular flow is formed in which the feed solution is continuously returned to the feed unit, or the flowing-out feed solution is introduced into a new alkaline cation separation apparatus. Thereby, the concentration of alkaline cations may be further reduced to a desired level. At this time, a new alkali cation separation apparatus may further separate alkaline cations through a diffusion dialysis device, an electrodialysis device, or dialysis or adsorption using an ion exchange material.

The recovery unit, which is a part located opposite the feed unit, receives the recovery solution through the recovery solution injection section. The alkaline cations that permeate and diffuse from the feed solution through the cation exchange membrane are dissolved in the recovery solution.

The recovery solution may be used without limitation as long as the recovery solution may facilitate the recovery of the alkaline cations. The recovery solution may be, for example, water or a solution mixture that is miscible with water and contains a non-solvent for the salts of the alkaline cations permeated through the cation exchange membrane into the recovery unit. The non-solvent is not limited thereto but may include acetone, isopropanol, ethanol, alkyl alcohol, and alkanolamine. The content of the non-solvent may be 0 wt % to 60 wt % or more based on the total amount of solvent mixture with water.

In the case of diffusion dialysis, diffusion based on the concentration difference of substances is used as a driving force for separation, so it is better to have a larger concentration difference for the substances to be transmitted around the ion exchange membrane if possible. Preferably, when the recovery solution is introduced into the recovery unit, alkaline cations derived from organic acid alkali salts in the recovery solution are substantially absent.

To achieve this, the recovery solution with a low concentration of alkaline ions is continuously injected into the recovery unit, and the recovery solution is stored in a separate container after diffusion dialysis. Alternatively, to achieve this, the separation apparatus may be configured to remove alkaline cations from the recovery solution after diffusion dialysis using a gas injection means, which will be described later, and then the resulting recovery solution circulates into the recovery solution injection section for reuse.

One embodiment of the present disclosure may further include a gas injection means for injecting at least one gas selected from the group consisting of carbon dioxide, sulfur dioxide, and nitrogen oxides into the recovery solution. In the embodiment, the gas injection is to inject a gas into the recovery solution. The configuration of the gas injection means is not limited as long as the gas may be well dispersed into the recovery solution. Generally, a bubbling method using a gas disperser such as a sparger is used.

In another embodiment, the gas injection means is to release the gas to the outside of the recovery solution and then inject the gas into the recovery solution using pressure. The pressure may be applied using a separate pressurizing means, or the pressure of the gas itself within the container may be used, but the pressurizing method is not limited thereto. However, to employ the gas injection means, the recovery unit or the container containing the recovery solution will need to be strong enough to withstand the pressure caused by the injected gas.

The gas injection means may be installed within the recovery unit or may be installed in a separate container where the recovery solution is stored after diffusion dialysis.

When at least one gas selected from the group consisting of carbon dioxide, sulfur dioxide, and nitrogen oxide is dissolved into the recovery solution by the gas injection means, the gas meets water in the recovery solution and, as a result, at least one anion such as carbonate ions, bicarbonate ions, sulfate ions, sulfite ions, nitrate ions, or nitrite ions are generated depending on the gas composition. At the same time, the gas/water mixture promotes the production of hydrogen cations. Additionally, when the concentration or content of the dissolved gas increases by increasing a flux of the gas injected into the recovery solution, the production of hydrogen cations is further promoted.

The generated anions cannot pass through the cation exchange membrane and remain in the recovery section, meanwhile, the hydrogen cations may pass through the cation exchange membrane and move to the feed unit where the hydrogen cations meet the organic acid anions remaining in the feed unit and are recovered as organic acids. That is, as a result of diffusion dialysis, imbalance in ionic charge that may occur due to the continuous movement of only alkaline cations to the recovery unit may be avoided in advance.

Meanwhile, the alkaline cations moving from the feed unit to the recovery unit combine with the carbonate anions remaining in the recovery unit to produce alkaline salts such as potassium bicarbonate, potassium carbonate, potassium sulfate, or potassium nitrate.

According to a further embodiment of the present disclosure, after diffusion dialysis, the alkali salts in the recovery solution may be easily removed by lowering their solubility and fixing the alkali salts in the form of a solid. To achieve this, a non-solvent may be added to the recovery solution. The non-solvent may be, for example, at least one of acetone or an alkyl alcohol, preferably at least one of acetone, ethanol, or isopropanol.

The removed alkali salts may be recycled in various fields. For example, potassium carbonate may be recycled in the organic acid salt production process, potassium sulfate may be used as a fertilizer, and potassium nitrate may be used as a fertilizer or gunpowder material.

The feed unit and the recovery unit may be further provided with a disturbance device to prevent the concentration gradient, which is a driving force of diffusion dialysis, from being lowered. The disturbance device is capable of causing an irregular fluid flow path and may operate with the use of a spacer or the like. The spacer has a grid shape, and as long as the spacer has a shape corresponding to a structure that can help form a turbulent flow, there are no limitations on the manufacturing method and shape of the spacer. The spacer may be manufactured by, for example, woven, nonwoven, knitted, or tricot methods.

The fluid flow direction of the feed unit and the recovery unit may be counter-current in opposite directions, or co-current in the same direction, as needed.

Since alkaline cations move from the feed unit to the recovery unit, the concentration of alkaline cations in the recovery solution may gradually increase. To prevent this increase in the concentration of alkaline cations, a removal means for removing alkaline cations from the recovery solution may be further provided.

The present disclosure provides a method for separating organic acid alkali salts into organic acids and alkali salts using an apparatus having a diffusion dialysis section.

The method of separating organic acid alkali salts into organic acids and alkali salts includes: injecting a feed solution containing organic acid alkali salts into the feed unit of an apparatus, which has a diffusion dialysis section divided into a feed unit and a recovery unit by a cation exchange membrane and injecting a recovery solution containing a solvent such as water and a non-solvent such as ethanol into the recovery section, and bubbling or pressurizing at least one gas selected from the group consisting of carbon dioxide, sulfur dioxide, and nitrogen oxides into the recovery unit, thereby, promoting diffusion dialysis of alkaline cations through the cation exchange membrane.

At this time, it is preferable to maintain the temperature of the diffusion dialysis section at 2° C. to 80° C. As long as the diffusion rate is not excessively slowed due to the temperature being within the temperature range, precipitation of the diffused alkali salt may be facilitated.

Additionally, a pH value of the recovery unit of the present disclosure may range from 2 to 7. In the pH range, the production of protons is promoted, and protons may easily permeate and diffuse into the feed unit, thereby promoting a balance of electricity in the feed unit. This may further improve the diffusion of alkaline cations.

Hereinafter, the content of the present disclosure will be described in more detail through examples and comparative examples, but the scope of the present disclosure is not limited by the following examples.

### EXAMPLE 1-3: CONFIGURATION OF DIFFUSION DIALYSIS APPARATUS

### Example 1

A batch-type diffusion dialysis apparatus divided into two compartments by placing and fixing one cation exchange membrane in the center was prepared. 500 mL of a solution mixture of potassium formate (1 M) and potassium hydroxide (2 M) was injected into a feed unit. As a recovery solution (permeate), deionized water was injected into a recovery unit at a rate of 1 L/min so that the deionized water flow was counter-current to a flow of the solution mixture in the feed unit. Then, Example 1 was prepared by bubbling carbon dioxide at a flux of 50 ccm using a gas injection device in the recovery unit.

### Example 2

Example 2 was prepared by performing the same method as in Example 1, except that potassium lactate (1 M) was used instead of potassium formate (1 M).

### Example 3

Example 3 was prepared by performing the same method as in Example 1, except that potassium gluconate (1 M) was used instead of potassium formate (1 M).

### Comparative Example 1

Comparative Example 1 was prepared by performing the same method as in Example 1, except that carbon dioxide was not bubbling in Example 1.

### Comparative Example 2

Comparative Example 2 was prepared by performing the same method as in Example 2, except that carbon dioxide was not bubbling in Example 2.

### Comparative Example 3

Comparative Example 3 was prepared by performing the same method as in Example 3, except that carbon dioxide was not bubbling in Example 3.

### <Experimental Example 1>Confirmation of Gas Bubbling Effect

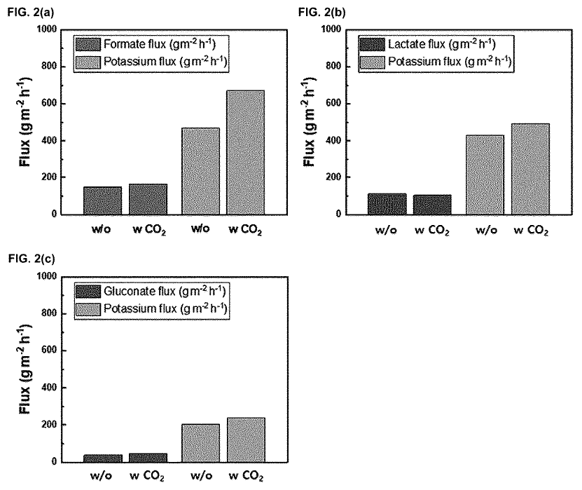

Diffusion dialysis according to Examples 1 to 3 and Comparative Examples 1 to 3 was performed for 20 hours. To measure a rate and efficiency of dialysis, a flux of any one organic acid anion selected from the group consisting of potassium ions, formate ions, lactate ions, and gluconic acid ions passing through the cation exchange membrane was measured in each of Examples 1 to 3 and Comparative Examples 1 to 3. The results are shown in Table 1 and FIG. 2 below.

In the table below, a refers to selectivity of ion permeation expressed as the permeability ratio of (K⁺/RCOO⁻).

TABLE 1

| | CO₂ | | | | | | | Gluconic acid | | |
|---|---|---|---|---|---|---|---|---|---|---|
| | Bubbling | Formic acid (C1) | | | Lactic acid (C3) | | | (C6) | | |
| | Status | | mol* | ms⁻¹ | | mol* | ms⁻¹ | | mol* | ms⁻¹ |
| Division | (◯/X) | g*m⁻²h⁻¹ | m⁻²h⁻¹ | (×10⁻⁶) | g*m⁻²h⁻¹ | m⁻²h⁻¹ | (×10⁻⁶) | g*m⁻²h⁻¹ | m⁻²h⁻¹ | (×10⁻⁶) |
| K⁺ | X | 469.8 | 12.0 | 6.73 | 430.0 | 11.0 | 6.85 | 205.3 | 5.3 | 4.18 |
| | ◯ | 671.1 | 17.2 | 9.36 | 494.1 | 12.6 | 7.50 | 239.4 | 6.1 | 5.01 |
| RCOO⁻ | X | 150.0 | 3.2 | 3.42 | 112.4 | 1.3 | 2.02 | 39 | 0.2 | 1.37 |
| | ◯ | 164.3 | 3.6 | 3.49 | 104.2 | 1.2 | 2.02 | 47.9 | 0.2 | 1.46 |
| α | X | 3.1 | 3.8 | 2.0 | 3.8 | 8.5 | 3.4 | 5.3 | 26.5 | 3.1 |
| | ◯ | 4.1 | 4.8 | 2.7 | 4.7 | 10.5 | 3.7 | 5.0 | 30.5 | 3.4 |

According to Table 1 and FIG. 2, it was confirmed that, due to the presence of the cation exchange membrane, positive potassium ions could easily pass through the recovery unit, meanwhile, diffusion of organic acid anions was suppressed. It was shown that the permeation of potassium ions through the cation exchange membrane was promoted by carbon dioxide bubbling.

The selectivity (α) of ion permeation could be obtained by the calculation formula 1 below. According to the calculation formula 1, it was confirmed that the selectivity increased by carbon dioxide bubbling and improved as the number of carbon atoms of the organic acid increased.

$$Selectivity ( \% ) = \frac{\begin{matrix}Potassium ion flux \\ ( Potassium flux , {gm}^{- 2} {h}^{- 1} )\end{matrix}}{\begin{matrix}Oragnic acid anion flux \\ ( Lactate flux {gm}^{- 2} {h}^{- 1} )\end{matrix}} 100 \qquad ( Calculation Formula 1 )$$

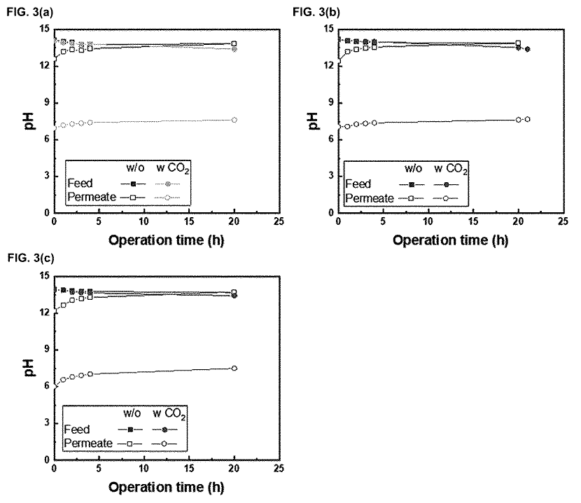

In addition, pH values of feed solution and recovery solution (permeate) in each of Examples 1 to 3 and Comparative Examples 1 to 3 were measured depending on dialysis time. The results are shown in FIG. 3.

Looking at FIG. 3, it was confirmed that, in Examples 1 to 3, the pH values of the recovery solution were maintained low by carbon dioxide bubbling, meanwhile, in Comparative Examples 1 to 3, the pH values of the recovery solution ranged between 12 and 13.5, which were very high compared to Examples 1 to 3. In the case of the feed solution, the pH values tended to decrease overall as dialysis progressed, and it was observed that decreases in the pH values were alleviated after 5 hours of dialysis.

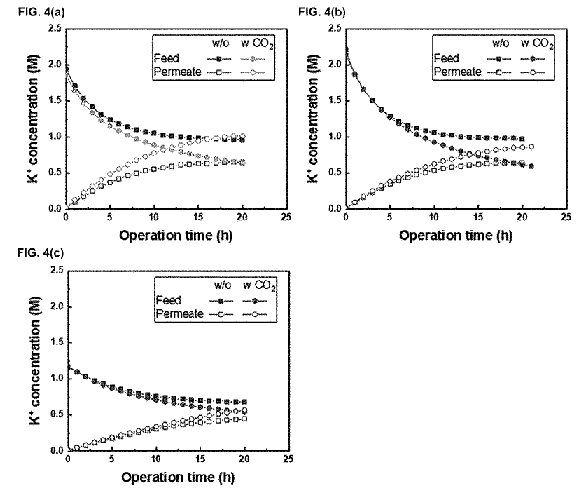

In addition, while performing diffusion dialysis according to Examples 1 to 3 and Comparative Examples 1 to 3, changes in ion concentration of feed solution and recovery solution (permeate) depending on dialysis time were measured in each of Examples 1 to 3 and Comparative Examples 1 to 3. The changes in potassium ion concentration were measured using a conductivity meter. The results are shown in FIG. 4.

According to FIG. 4, similar changes in potassium ion concentration were confirmed overall, but it was confirmed that the permeability of potassium ions gradually increased as CO₂ gas was added. This was believed to result from an efficient establishment of concentration equilibrium, driven by charge balance, as HCO₃⁻ ions generated by the formation of carbonic acid by CO2 gas combine with K⁺ while H⁺ permeates into the feed unit.

### <Experimental Example 2>: Alkaline (Bi)Carbonate Precipitation Experiment by Addition of Non-Solvents

To test a precipitation effect of alkali (bi)carbonate depending on the type and concentration of non-solvents, the recovery solution obtained in Example 1 (potassium bicarbonate, 500 mL KHCO₃ 1 M, FIG. 4(a)) was divided into 20 ml vials. Samples were prepared in which acetone, isopropanol, and ethanol each were added as exemplary non-solvents at a concentration of 20, 30, 40, 50, and 60 wt %, respectively. After mixing each of the solutions with a homogenizer until each of the solutions became uniform. Then, the present inventors waited for the generated KHCO₃ to settle. After settling, a decrease in conductivity of each of the solutions was measured to back-estimate the amount of K⁺ in the solution that had precipitated as solids of KHCO₃. Then, the amounts of solids obtained were compared.

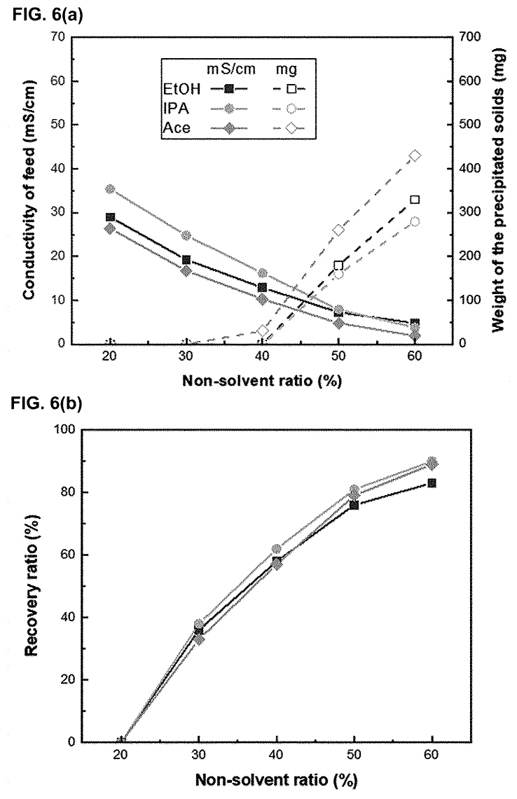

As shown in FIG. 6, when 40% or more of each of the exemplary non-solvents was added, it was confirmed that KHCO₃ was efficiently precipitated and obtained. It was found that the amount of KHCO₃ obtained was the same as the amount estimated from a dried weight and conductivity reduction. Therefore, it was found that up to 90% of KHCO₃ could be obtained as solids in a recovery solution through the addition of a non-solvent. This allowed for maintaining a K⁺ concentration in the recovery unit at an existing 10% level. Thus, it was expected to maintain a large concentration difference, thereby increasing a driving force.

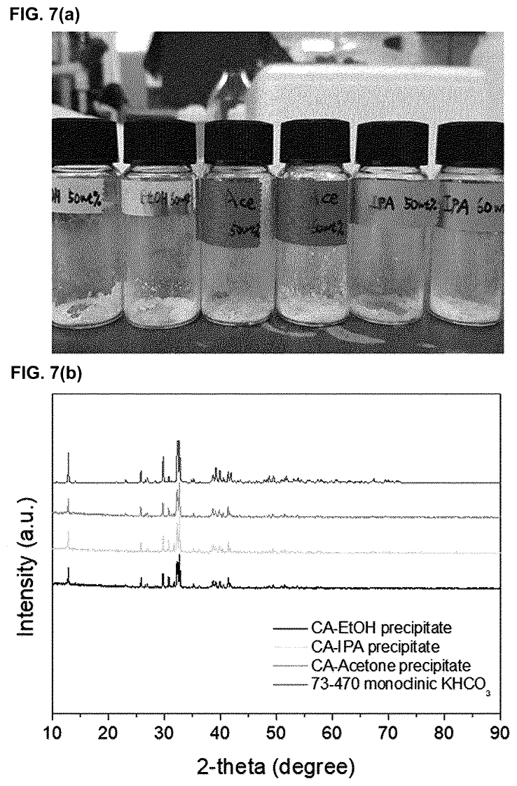

In addition, to confirm whether the solids obtained through the non-solvent addition experiments were KHCO₃, XRD analysis was performed as shown in FIG. 7. As shown in FIG. 7, it was confirmed that the diffraction pattern matched that of KHCO₃.

Although the preferred embodiment of the present disclosure has been described as above, the present disclosure is not limited thereto. The present disclosure can be implemented with various modifications within the scope of the claims, the detailed description of the present disclosure, and the accompanying drawings. It is natural that this also falls within the scope of the present disclosure.

### Industrial Applicability

The present disclosure relates to an apparatus and method for separating organic acid alkali salts into organic acids and alkali salts using the principle of diffusion dialysis. The present disclosure discloses an energy-efficient and environmentally friendly separation apparatus and method for utilizing gases corresponding to greenhouse gases or exhaust gases in a diffusion dialysis system, and thus industrial applicability of the apparatus and method is recognized.

1. An apparatus separating an organic acid alkali salt into an organic acid and an alkali salt, the apparatus comprising:
a diffusion dialysis section divided into a feed unit and a recovery unit by a cation exchange membrane;
a feed injection section for injecting a feed solution containing an organic acid alkali salt into the feed unit;
a recovery solution injection section for injecting a recovery solution containing water into the recovery unit; and
a gas injection means for injecting at least one gas selected from the group consisting of carbon dioxide, sulfur dioxide, and nitrogen oxide into the solution in the recovery unit.

2. The apparatus of claim 1, wherein the organic acid contains at least one of straight chain and branched chain hydrocarbons with 1 to 30 carbon atoms, monocyclic or polycyclic hydrocarbons with 6 to 30 carbon atoms, and aromatic hydrocarbons with 6 to 30 carbon atoms in a structure thereof.

3. The apparatus of claim 1, wherein the cation exchange membrane has at least one functional group selected from the group consisting of —COOH, —OH, and —SO₃H.

4. The apparatus of claim 1, wherein a means for disturbing a fluid flow is further provided in the feed unit and the recovery unit, respectively.

5. The apparatus of claim 1, wherein fluid flow directions in the feed unit and the recovery unit are opposite to each other or are the same.

6. The apparatus of claim 1, wherein the apparatus is further provided with a separation means for separating the alkaline salt generated in the recovery unit into a recovery solution.

7. A method of separating an organic acid alkali salt into an organic acid and an alkali salt, the method comprising:
injecting a feed solution containing an organic acid alkali salt into a feed unit of an apparatus, which has a diffusion dialysis section divided into the feed unit and a recovery unit by a cation exchange membrane and injecting a recovery solution containing water into the recovery unit, and
injecting at least one gas selected from the group consisting of carbon dioxide, sulfur dioxide, and nitrogen oxides into the recovery unit, thereby, promoting diffusion dialysis of an alkaline cation through a cation exchange membrane.

8. The method of claim 7, wherein the organic acid contains at least one of straight chain and branched chain hydrocarbons with 1 to 30 carbon atoms, monocyclic or polycyclic hydrocarbons with 6 to 30 carbon atoms, and aromatic hydrocarbons with 6 to 30 carbon atoms in a structure thereof.

9. The method of claim 7, wherein the method further comprises recovering the alkali salt diffused into the recovery unit from the recovery solution.

10. The method of claim 7, wherein the gas is carbon dioxide, and
the alkali salt diffusing into the recovery unit is alkali carbonate and/or alkali bicarbonate.

11. The method of claim 7, wherein the diffusion dialysis section has a temperature in the range of 2° C. to 80° C.

12. The method of claim 7, wherein the recovery unit may have a pH in the range of 2 to 7.
